(19) United States
(12) Patent Application Publication
(10) Pub. No.: US 2024/0038348 A1
(43) Pub. Date: Feb. 1, 2024

(54) HEALTH CARE MONITOR

(71) Applicant: ANTONIO ANGELO RESTIVO, II, PARADISE VALLEY, AZ (US)

(72) Inventor: ANTONIO ANGELO RESTIVO, II, PARADISE VALLEY, AZ (US)

(21) Appl. No.: 17/877,931

(22) Filed: Jul. 30, 2022

(51) Int. Cl. G16H 15/00; G16H 20/00; G16H 40/67; A61B 5/00; A61B 5/02

(52) U.S. Cl. CPC G16H 15/00; G16H 20/00; G16H 40/67; A61B 5/4848; A61B 5/02; A61B 5/743; A61B 5/0002

(57) ABSTRACT

A health monitoring system is described that permits daily tracking of medical conditions, journaling of information pertaining to treatments of the conditions, and providing a graphical user interface to see overall progress related to the conditions. The monitoring system includes a conditions schema, a journaling schema, and a user schema. The user schema accesses a conditions table generated by the conditions schema and a separate journal table to generate graphical representations.

### FIELD OF THE INVENTION

The present invention relates to health care monitoring systems, in general, and to health care monitoring systems and methods for individuals having health issues, in particular.

### BACKGROUND

Some people have medical issues and symptoms or medication/treatment schedules that need to be monitored daily. For example, individuals having covid, vaccination reaction, stroke, hypertension, as well as separate health conditions that plateau due to ineffective treatment plans need to be monitored daily. Caretakers, doctors, or patients may not have an organized methodology to track patient conditions or may not be readily able to determine the effectiveness of a prescribed treatment plan and/or may not be have the ability to easily compare the effectiveness of a given medication or treatment plan to a prior medication or treatment plan.

In addition, patients, caretakers, and doctors may not currently be able to properly identify the frequency of symptoms or possible triggers of these symptoms due to either the lack of independent tracking, or due to having incomplete or disorganized records or records that are not effectively summarized.

The failure to properly identify the frequency of symptoms or possible triggers of symptoms may result in incorrect diagnoses, ineffective treatment methods and medication, unrecognized symptoms, patient strain, increases in treatment time and resource costs, or even a decrease in motivation to seek help for a chronic condition.

### SUMMARY

The present invention provides a health care monitor system and method that allows a patient or caretaker user to utilize daily tracking of multiple conditions and medications to provide organized metrics and charts for easier analysis and comparison of treatment methods.

The present invention fills a major gap in the health care and patient treatment industry by giving clarity, definition, and metrics to the ambiguities in real world care. These health care ambiguities include quality of care, frequency of symptoms or side effects, and response to a given treatment, amongst many others. These ambiguities most commonly result from second-hand accounts, recalled memories, or estimations not based on tangible data.

An embodiment of a health monitoring system permits daily tracking of medical conditions, journaling of information pertaining to treatments of the conditions, and providing a graphical user interface to see overall progress related to the conditions comprises a processor, a display coupled to the processor, input apparatus for providing inputs to the processor, a wireless interface for providing wireless access to a network, a program memory coupled to the processor, and a non-transient memory accessible by the processor, The non-transient memory comprises program instructions executable by the processor. The program instructions comprise a conditions schema, a journal schema, and a user schema.

The system comprises a data tracking database and a conditions database.

The conditions schema comprises instructions to receive, via the input apparatus, entered health conditions, commentary on the patient's conditions, and commentary on treatment for the conditions. The conditions schema stores the health conditions, patient condition commentary, and treatment commentary in the conditions database and generates a conditions table in a page. The conditions page comprises a treatment plan and a retrospect of past treatments.

The journaling schema comprises instructions for receiving daily journaling entries in the data tracking database and generates a journaling page in the data tracking database.

The user schema comprises instructions to automatically generate graphical representations from the conditions schema and the journals schema and displays the graphical representations on the display upon request from a system user.

In the embodiment, user schema instructions executable by the processor utilize the data tracking database and the conditions database to track user consistency with the treatment plans and to generate a graphical representation of user consistency. The processor is operable to display the graphical representation of user consistency on the display.

In the embodiment, the processor is operable to transmit the graphical representation of user consistency via the wireless interface.

In the embodiment, the user schema instructions are executable by the processor to automatically generate a graphical representation showing effectiveness of the treatment plans.

In the embodiment, the processor is operable to selectively transmit the graphical representation showing effectiveness and the graphical representation of user consistency via the wireless interface.

In the embodiment, the user schema instructions are executable to automatically generate and visually display graphical representations of the conditions and treatment progression. The graphical representations show how the patient's condition is changing and providing visualization of amelioration of improvements and quantifiable aspects of the journal entries and the conditions.

In the embodiment, the journaling schema instructions are executable to receive and store user created variables within the journaling page and the processor is operable to display the user created variables. The user schema instructions are executable to create one of more graphical representations of the user created variables.

In the embodiment, the user schema comprises instructions executable to permit a user to check whether one of one or more health conditions is improving or not and comprises instructions to generate graphical representations from the user selected variables.

In the embodiment, the program instructions are executable on the processor to track an index of the conditions in the conditions page separate from the daily journaling page.

In the embodiment, the program instructions are executable to display a list of conditions that a user can refer to with respect to a treatment plan and its effectiveness.

In the embodiment, the executable program instructions generate one or more tables from a union of the conditions table and the journaling table to permit determining which of the conditions are improving.

In the embodiment, the conditions schema provides a ranking scale responsive to the user to rank a treatment, and the program instructions utilizes the ranking scale to assist the user to determine whether or not a new treatment plan is needed.

In the embodiment, the journaling schema comprises program instructions executable to be responsive to the user entering a description of the treatment plan to store and display the treatment plan. The program instructions are responsive to the user entering one or more links to images and videos of the condition to store and display the images and videos.

In the embodiment, the system combines functionality from the journal schema, the conditions schema, and the user schema to automatically produce graphical representations from the conditions schema.

In the embodiment, the graphical representations include automatically generated graphs of user consistency, of patient blood pressure, and of user selected variables, and the processor provides a graphic user interface to visualize the graphical representations.

In another embodiment, a non-transient memory comprising program instructions executable by a processor to provide a health monitoring system for a patient that permits daily tracking of medical conditions and journaling of information pertaining to treatments of the conditions and providing a graphical user interface to see overall progress related to the daily tracking and journaling is provided. In the another embodiment, the program instructions comprise a conditions schema, a journal schema, and a user schema. The another embodiment comprises a data tracking database for use by the processor and a conditions database for use by the processor. In the another embodiment, the conditions schema includes instructions to receive, via the input apparatus, entered health conditions, commentary on the patient's conditions, and commentary on treatment for the conditions. The conditions schema stores the health conditions, patient condition commentary, and treatment commentary in the condition database and generates a conditions table in a separate page. The conditions page comprises a treatment plan and a retrospect of past treatments. The journaling schema comprises instructions for receiving daily journaling entries in the data tracking database and generating a journaling page in the data tracking database. The user schema comprises instructions to automatically generate graphical representations from the conditions schema and the journals schema and displaying the graphical representations on a display upon request from a system user.

In the another embodiment, the user schema instructions are executable by the processor to utilize the data tracking database and the conditions database to track user consistency with the treatment plans and to automatically generate a graphical representation of user consistency.

In the another embodiment, the user schema instructions are executable by the processor to automatically generate a graphical representation showing effectiveness of treatment plans.

In the another embodiment, the user schema instructions are executable to automatically generate graphical representations of the conditions and treatment progression. The graphical representations show how said patient's condition is changing and provide visualization of amelioration of improvements and quantifiable aspects of the journal entries and conditions via a graphical user interface.

In the another embodiment, the journaling schema instructions are executable to receive and store user created variables within the journaling page, and the user schema instructions are executable to create one of more graphical representations of user created variables.

In the another embodiment, the user schema comprises instructions executable to permit a user to determine whether one of the one or more health conditions is improving or not and comprises instructions to generate graphical representations from user selected variables.

In the another embodiment, the program instructions are executable on the processor to track an index of conditions in the conditions page separate from daily journaling page.

In the another embodiment, the program instructions are executable to display a list of conditions that a user can refer to with respect to a treatment plan and its effectiveness.

In the another embodiment, the executable program instructions generate one or more tables from a union of the conditions table and the journaling table to permit determining which conditions are improving.

In the another embodiment, the conditions schema provides a ranking scale responsive to the user to rank a treatment and the program instructions utilize the ranking scale to assist the user to determine whether or not a new treatment plan is needed.

In the another embodiment, the journaling schema comprise program instructions executable to be responsive to the user entering a description of the treatment plan to store and display the treatment plan, and the program instructions are responsive to the user entering one or more links to images and videos of condition to store and display the images and videos.

In the another embodiment, the graphical representations include automatically generated graphs of user consistency, of patient blood pressure, and of user selected variables, and the processor provides a graphic user interface to visualize the graphical representations.

### BRIEF DESCRIPTION OF THE DRAWING

The invention will be better understood from a reading of the following detailed description in conjunction with the drawing figures in which like elements are given like designators and in which the sizes and shapes of various display elements are exemplary only and in which:

FIG. 1 is a block diagram of a system; and

FIGS. 2 through 16, inclusive, are images of graphical user interface displays.

### DETAILED DESCRIPTION

The present invention fills the above-described major gap in health care and patient treatment through a system and method that allows for tracking multiple universal health issues.

In the description that follows, the term “condition” is used as a place holder for real health conditions. Alternatively, the name of the condition can represent real health conditions such as, for example, “covid”, “vaccination reaction”, “cancer”, “spider veins”, “gastric reflux”, etc.

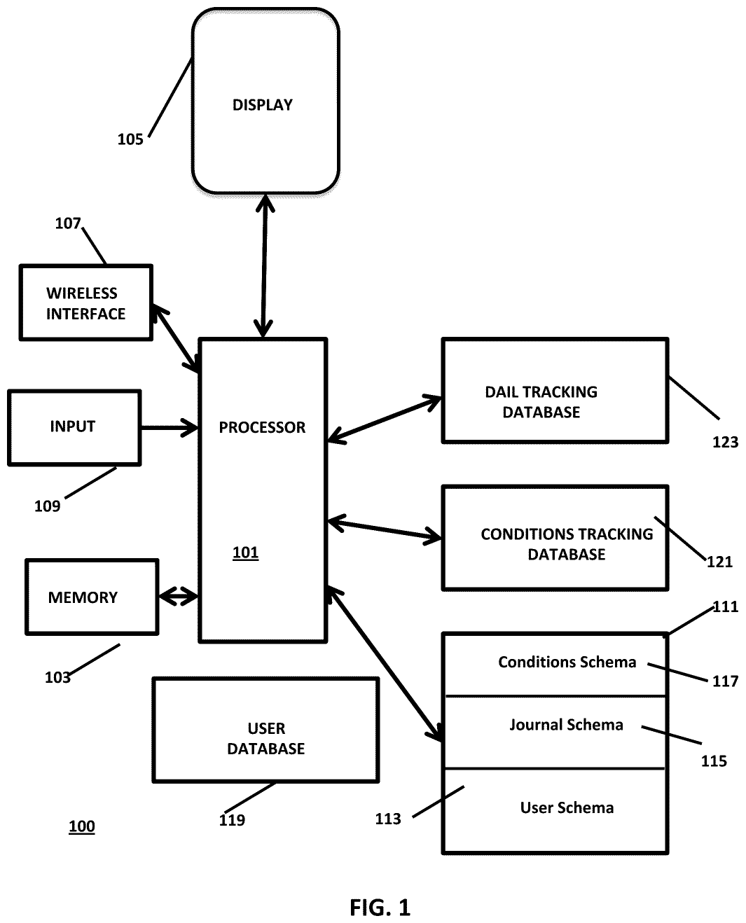

Turning to FIG. 1, system 100 comprises a processor or microprocessor 101, memory 103 including an operating system, and a display 105. System 100 also includes a wireless interface 107. Wireless interface 107 can be a Wi-Fi interface that provides access to a local area network, virtual private network, and/or to the Internet, none of which are shown in the drawing for clarity. Input apparatus 109 is also provided. Input apparatus 109 can be any conventional input apparatus including a keypad, buttons, voice input and a touch screen on display 105.

System 100 also comprises module 111. Module 109 comprises non-transient memory that includes a health monitor program. Health monitor program 111 includes a user schema 113, daily tracking schema 115, and conditions tracking schema 117. User schema 113 utilizes a user database 119 that may be a dedicated database or a portion of memory 103 or in other embodiments may be hosted separately from system 100. Daily tracking schema 115 supports a daily tracking database table 121 and conditions tracking schema 117 supports conditions tracking database table 123 each database table may be in a separate memory, or in a portion of memory 103, or hosted separately from system 100.

Processor 101 in response to health monitor program 111 may display two separate sets of pages. One set of pages is associated with daily tracking database table 121 and another set of pages is associated with conditions tracking database table 123. A user of system 100 is given access to both database tables 121, 123 and the associated sets of pages. This methodology allows for tracking of multiple universal health issues.

In one embodiment that is described herein, a patient was diagnosed with suffering a stroke resulting from hypertension. A caretaker may journal variables that occur during each day and include pictures of tongue, description of health routines, description of the day in general, and annotations related to blood pressure to enhance blood pressure tracking and dietary changes. It should be noted that the following description is specific to the stroke patient, but as will be clearly understood by those of ordinary skill in the art, the invention is not limited in application to stroke victims. System 100 is applicable to patients suffering from many other healthcare issues including, but not limited to: Covid, vaccination reactions that are not uncommon with Covid, cancer, infections and many other health conditions.

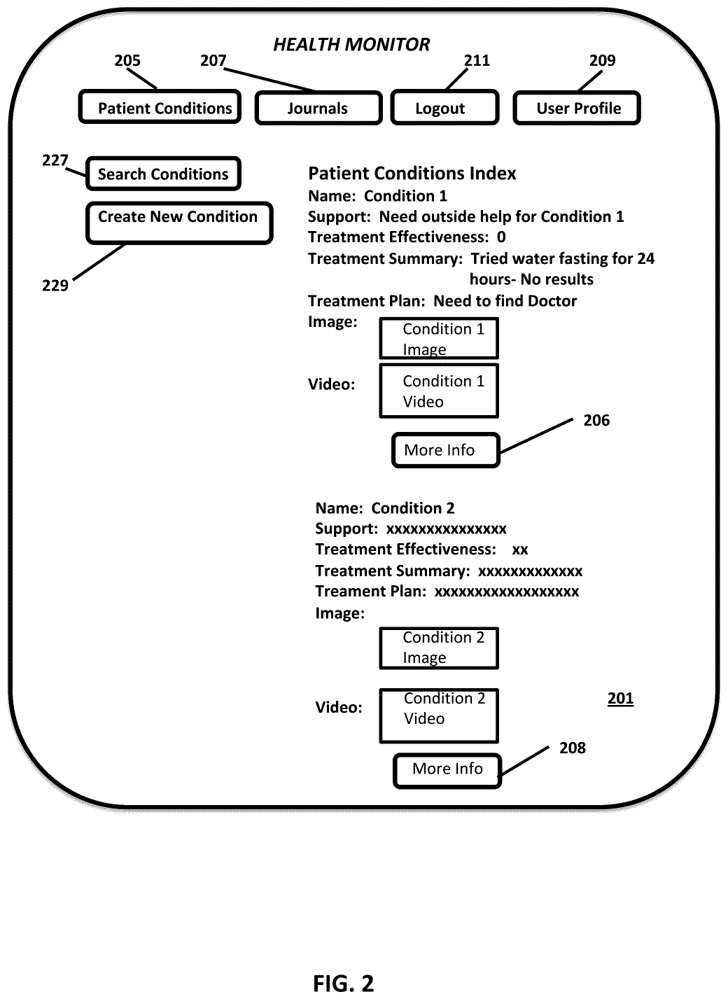

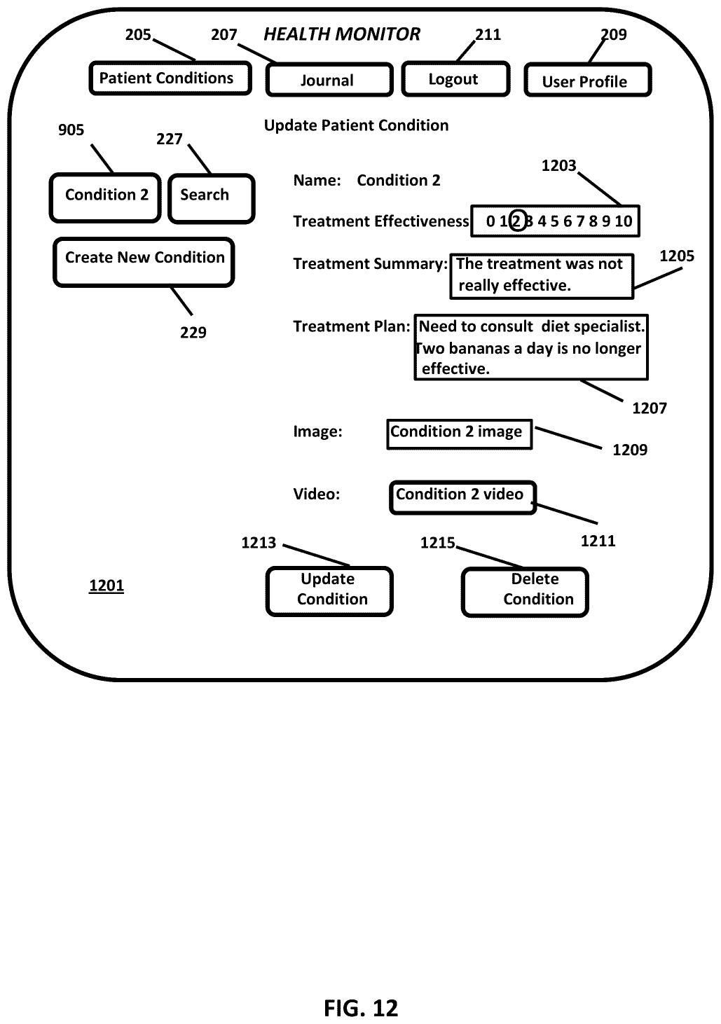

System 100 is used to create daily entries for a patient. In the description set forth herein below of FIGS. 2 through 16, inclusive, various pages for entry of patient information as generated by system 100 are described. The information entered by a user includes a daily journal. To create a new journal, a New Journal button is used. The new journal button when clicked causes system 100 to display a New Journal page. The New Journal page includes a description text field for providing descriptive information. In addition, the user can enter an image ink and a video link to upload an image of a condition and a video of the condition. Furthermore, health routines, blood pressure average and annotations may be entered. In this embodiment, an image of the patient's tongue may be loaded.

A New Metric button is clicked to add new metric variables. Clicking on the New Metric button causes system 100 to display a page for adding a new metric to the daily journal.

The user clicks submit to input the data for the day. To access any journal the user would click More Info to see the entire journal archive recorded that day. The user would see when the journal was recorded, description, health routines/diet, and blood pressure average for the day, blood pressure annotations, image, video, image of tongue, and the user generated metrics.

System 100 also provides an option to update the journal entry for a day by clicking on an Update Journal button. Within the Update Journal page there is also the option to delete the journal for the day as well.

A Patient Conditions button is provided on pages that are displayed. When the user clicks the Patient Conditions button, the user has access to all the conditions the user has made as well as a Create New Condition button to create a new condition.

When the user clicks the “Create New Condition” button the user has access to empty string/text fields and a Boolean check. The user can input the condition name, input the 1-10 number to rank the treatment plan. The user can also input a treatment retrospect, a treatment plan, image link, and video link.

After entering the new condition inputs the user can click a Submit button to add a new condition. To access any condition in its entirety the user clicks a More Info button to see the condition in its entirety. When the user clicks the More Info button the user System 100 displays a page that shows the time when the condition is recorded, if the condition needs outside support, a treatment retrospect, a treatment plan, video, and an image. A Return button when clicked returns the user to the conditions list, and Update Condition button is clicked to update the condition.

When the user clicks the Update Condition button System 100 displays all the prefilled data in the textbox/string fields. The user may edit the prefilled data or delete the entire condition itself via a Delete Condition button—with a confirmation button before deleting.

A conditions table is provided in a separate page from the daily tracking/journaling table. Utilizing the conditions table, the user can list other conditions that may not come to the caretakers/patients mind during daily tracking.

Conditions listed in the conditions table may include a variety of conditions, such as by non-limiting example, finger nail fungus. In addition, a treatment plan and retrospect of past treatments may be added so the user can check later if the treatment plan works or if the patient needs to try something different. The user can also see the past images or videos of the finger nail fungus to see if the treatment plan works.

The user can also list other chronic conditions as well, such as lethargy or ataxia (a byproduct of the stroke).

On display 105, patient conditions and treatment progression may be visually displayed graphically. Graphical displays are useful show how the patient's condition is changing and permits the visualization of amelioration of improvements with the quantifiable aspects of the Journals and Conditions tab. For example, if tracking blood pressure, daily numbers from the Journal application will be charted in a graph versus timeline.

A graph for tracking user consistency with the treatment plans may also be displayed.

The various pages displayed on display 105 help the user see what treatment plans have been most effective. The displays include a graph showing the effectiveness of treatment plans for the patient.

In the embodiment of the invention, system 100 automatically creates one or more graphs of any user created variables.

The foregoing summarizes how system 100 executing program instructions stored in a non-transient memory can address the issues a user or doctor may have in visualizing and tracking the effectiveness of treatment plans and conditions a patient may have and to help the doctor and patient take a better course of action in pursuing treatments.

The system provides tracking an index of conditions related to a patient in one page separate from daily journaling; the ability for the user to create new variables within a journals tab; the ability to check whether a health condition is improving or not, and seeing graphs of all custom user variables, as well as default user variables on blood pressure, treatment effectiveness, and user consistency.

The daily journaling schema is universal to all patients. The user may place custom variables into the journaling schema making it more universal. Different data elements can be changed or the user can change them for daily journaling and keep daily tracking and conditions monitoring in two separate data file location. Having separate conditions and daily tracking pages helps the user avoid forgetting conditions that may otherwise be forgotten in daily tracking. The conditions page allows a list of conditions that a user can refer to with respect to a treatment plan and its effectiveness. One or more tables stemming from both a union of both the conditions table and daily journaling table are provided to see what conditions are improving from the routines in the daily journaling table.

The conditions schema provided is particularly very useful and original in that the user can rank a treatment with a ranking scale. In the embodiment the ranking scale is a rank from 1 through 10. Utilizing the ranking scale, the system determines whether the user needs a new treatment plan or not.

The treatment retrospect text box permits the user to reflect on whether a past treatment plan has worked.

A treatment plan text box is provided. The user can fill in a description of the treatment plan along with one or more images and videos of the condition.

Providing summarized and organized graphs of all custom user variables as well as included variables of blood pressure, treatment effectiveness, and user consistency with respect to time are particularly advantageous. These let the user as well as a doctor to see the data in an organized way without having to scroll through potentially dozens of entries throughout the day. This could help a doctor as well as the user to see overall progress related to the daily routines.

The system of the embodiment comprises functionality from journal schema, conditions schemas, and user schema and graphical output that is automatically produced from the conditions schema.

The journal schema is non-specific for any health condition and helps the user look at the patient as a whole as well as add or track any other variables on a daily basis.

The conditions schema helps the user isolate a given health condition that the patient has and to see whether or not the condition is improving with the treatment plan or not.

The user schema includes graphs that are auto-generated over time from both the patient conditions schema and the journals schema, and includes graphs of user consistency, of patient blood pressure, and of all custom user variables. This helps the user or doctor see results in a graphic users interface (GUI) and to help motivate the user to use the embodiment.

A user begins with a log in or sign up form that is displayed on display 105 by processor 101 executing health monitor program 111. The log in and sign up form may be of a type that is known in the art. In this embodiment, the sign up form only requires an email address, password, and password confirmation. The login form requires an email and password.

After a user signs up or logs in, system 100 displays Patient Conditions Index page 201 shown in FIG. 2. At the top of page 201 and all other pages that are described herein below are displayed are Patient Conditions button 205, Journals button 207, and User Profile button 209. A logout button 211 is also displayed.

Page 201 is shown for a user that has already entered Patient Conditions and displays the entries for those patient conditions. Shown are the entries for Condition 1 and Condition 2. Although only two conditions are shown in FIG. 2 for clarity, several conditions may be monitored. By scrolling down, additional conditions for the patient, if any, are displayed.

Each condition entry displayed identifies the condition, shows whether support is needed, the effectiveness for the treatment on a scale of 0 through 10, a summary of the treatment, the treatment plan going forward, and uploaded images of the condition, as well as uploaded videos of the conditions. Each condition summary includes a More Info button 206, 208 that is used to obtain more information that has been entered for the respective condition.

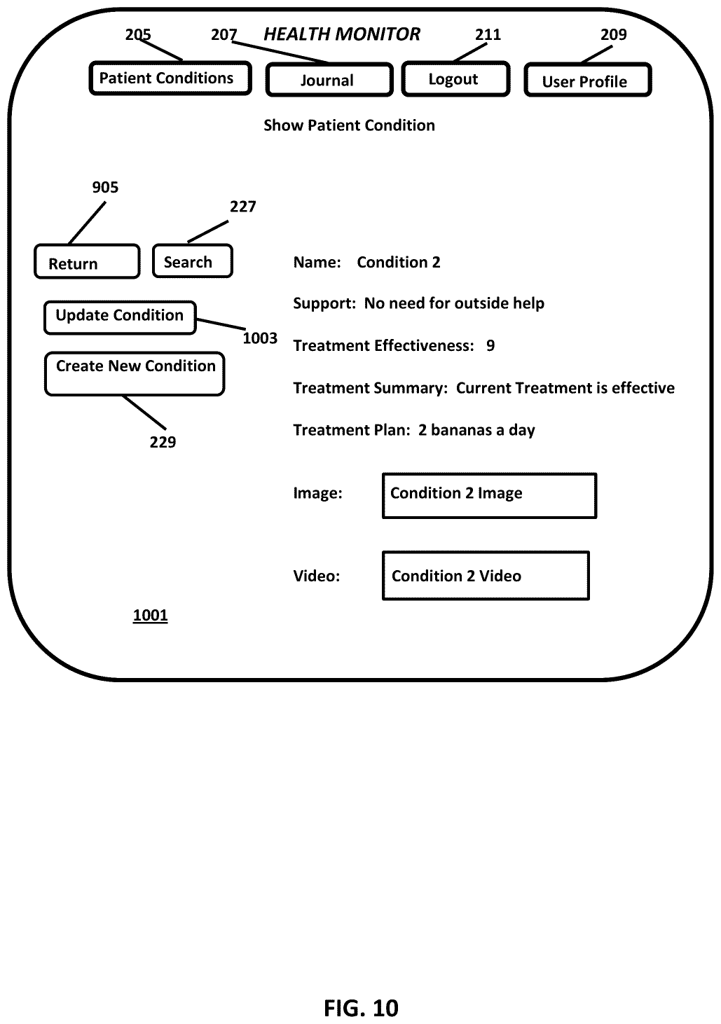

For example, if more information is desired for Condition 2, More Info button 208 may be clicked and more details for the condition are shown on page 1001 shown in FIG. 10. Page 1001 displays information entered by the user with respect to the condition. In this instance, a description of the treatment is provided along with an indication that the treatment does not need any outside support, In other instances, there may be an indication that that the treatment plan is not effective and/or that outside support is needed to treat the condition. The treatment plan that has been entered by the user is also displayed. If an image and/or video of the condition has been uploaded, that video and/or image may also be viewed. By clicking on Update Condition button 1003, the user can update the condition information. By clicking on Return button 905, system 100 will return to page 201, shown in FIG.2.

In addition, a Search Conditions button 227 and a Create New Conditions button 229 are provided.

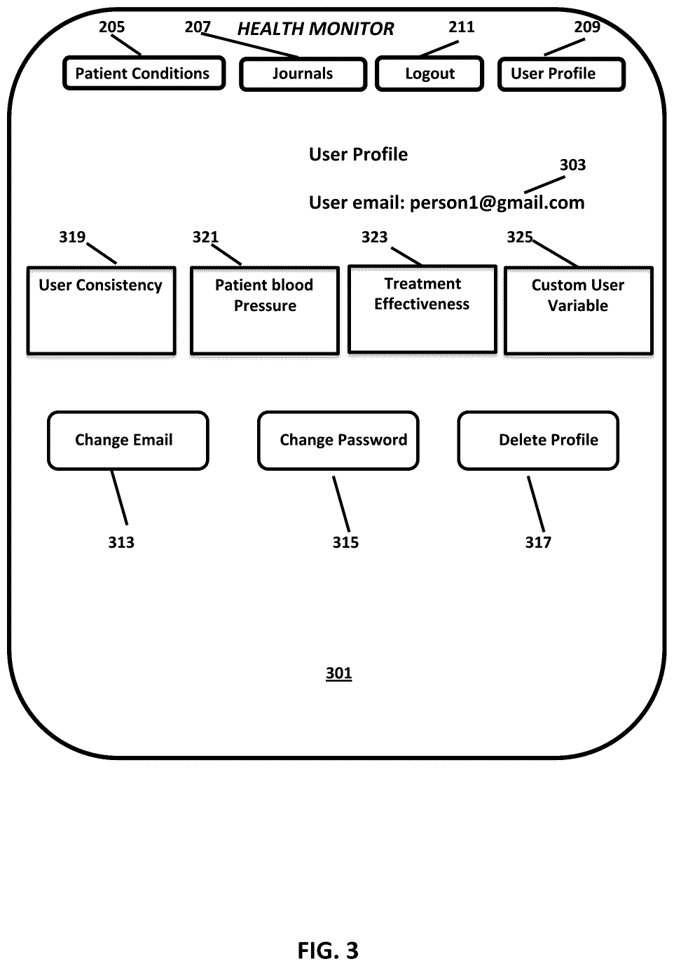

If the user clicks on User Profile button, the use's profile page 301 shown in FIG. 3 which shows the user's email address 303. Page 301 additionally displays a Change Email button 313, Change Password Button 315, and a Delete Profile button 317.

When Change Email button 313 is clicked, another page is displayed, which is not shown for clarity and brevity, on which the email address of the user may be changed.

When Change Password button 315 is clicked, a further page is displayed, which is not shown for clarity and brevity, on which the user password may be changed.

When Delete Profile button 317 is clicked, a page is displayed, which is not shown for clarity and brevity, on which the user profile may be deleted.

Page 301 additionally displays User Consistency button 319, Patient Blood Pressure button 321, Treatment Effectiveness button 323, and Custom User Variable button 325.

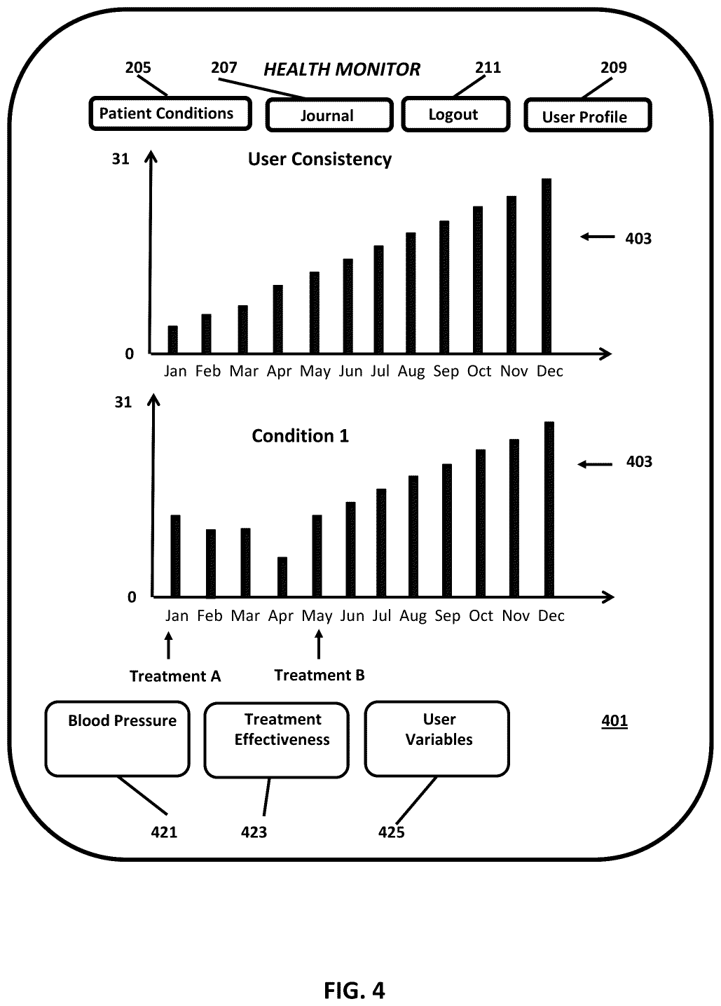

Clicking on User Consistency button 319 results in system 100 causing page 401 shown in FIG. 4 to be displayed.

Page 401 displays a User Consistency graph 403 and Conditions Tracking graphs 405.

User Consistency graph 403 shows in graphical form how consistently the user uses the daily journal and accommodates months having 28, 29, 30, and 31 days. Although graph 403 is shown as a bar graph, it will be understood that different forms of graphs may be used and displayed.

Conditions Tracking graphs 405 include graphs for all user conditions. Only one graph 307 is shown and if there are additional Condition Tracking graphs 405, those graphs may be viewed by scrolling down page 401. Each Condition Tracking graph 405 displays a graph tracking how conditions are tracked and/or updated on a daily basis. Each Condition Tracking graph also provides an indication when a treatment plan begins and ends. A treatment changes when a treatment plan is changed. The first treatment plan is identified as Treatment A and when the treatment plan changed it is identified as Treatment B. Further changes are similarly identified.

Although graph 407 is shown as a bar graph, it will be understood that different forms of graphs may be used and displayed for Condition Tracking graphs 405.

Page 401 includes Blood Pressure button 421, Treatment Effectiveness button 423, and User Variables button 425.

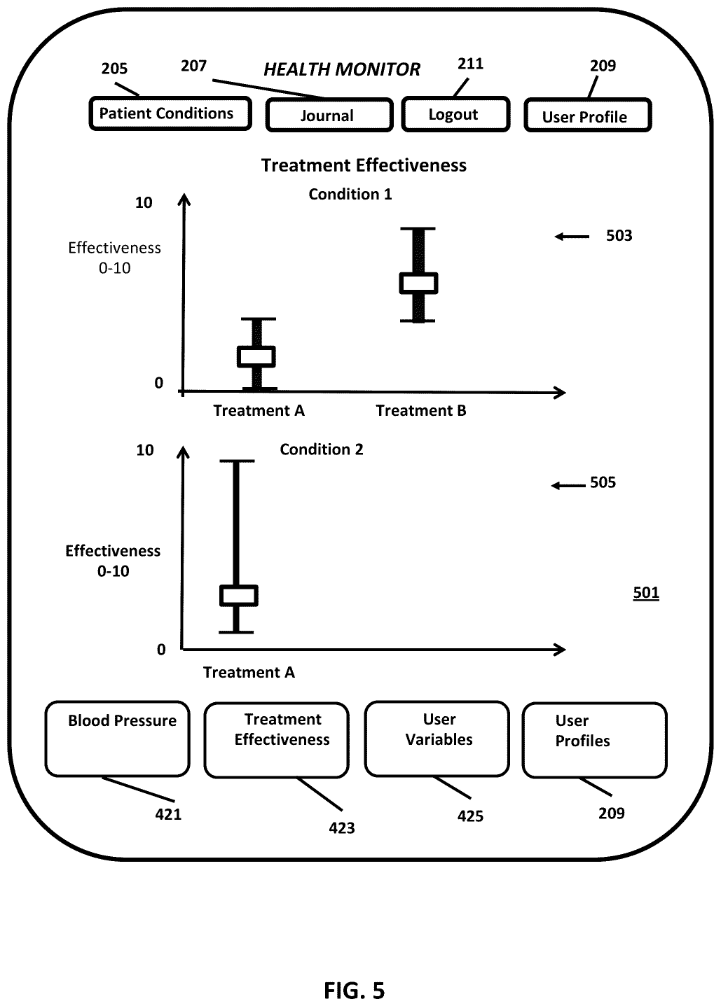

Clicking Treatment Effectiveness button 423 results in page 501 shown in FIG. 5 being displayed. Page 501 shows treatment effectiveness graphs for each condition. For Condition 1, treatment effectiveness graph 503 is shown. For condition 2, treatment effectiveness graph 505 is shown. Although only two conditions are shown, by scrolling down page 501 additional conditions, if any, will be displayed.

Each condition graph displays treatment effectiveness rated from 0 through inclusive, with 10 indicating the most effective treatment and 0 indicating an ineffective treatment. The length of each line for a treatment shows its effectiveness over time. Graph 503 shows the effectiveness for two treatments, i.e., treatment A and treatment B, for condition 1.

Graph 505 shows the effectiveness of one treatment for Condition 2.

The treatment effectiveness is archived ranks from a conditions file shown on a conditions page described herein below.

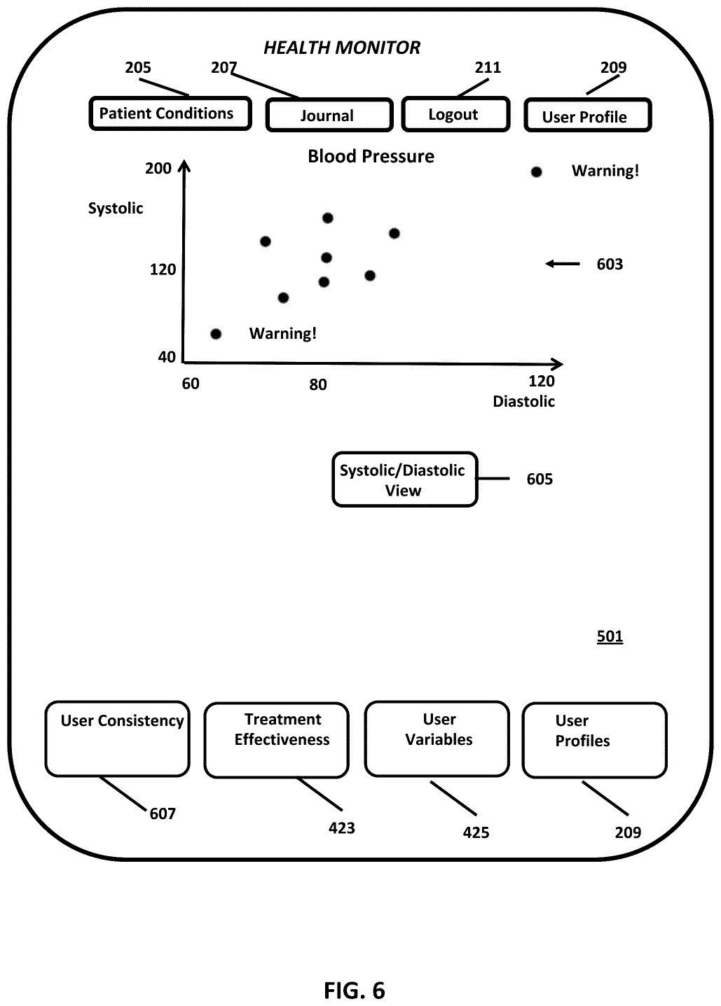

If blood pressure button 421 is clicked, page 601 shown in FIG. 6 is displayed. Page 601 presents a graph of all blood pressure readings plotted with systolic readings on the vertical axis and diastolic on the horizontal axis. Any blood pressure readings that are outside normal limits are flagged with “warning”.

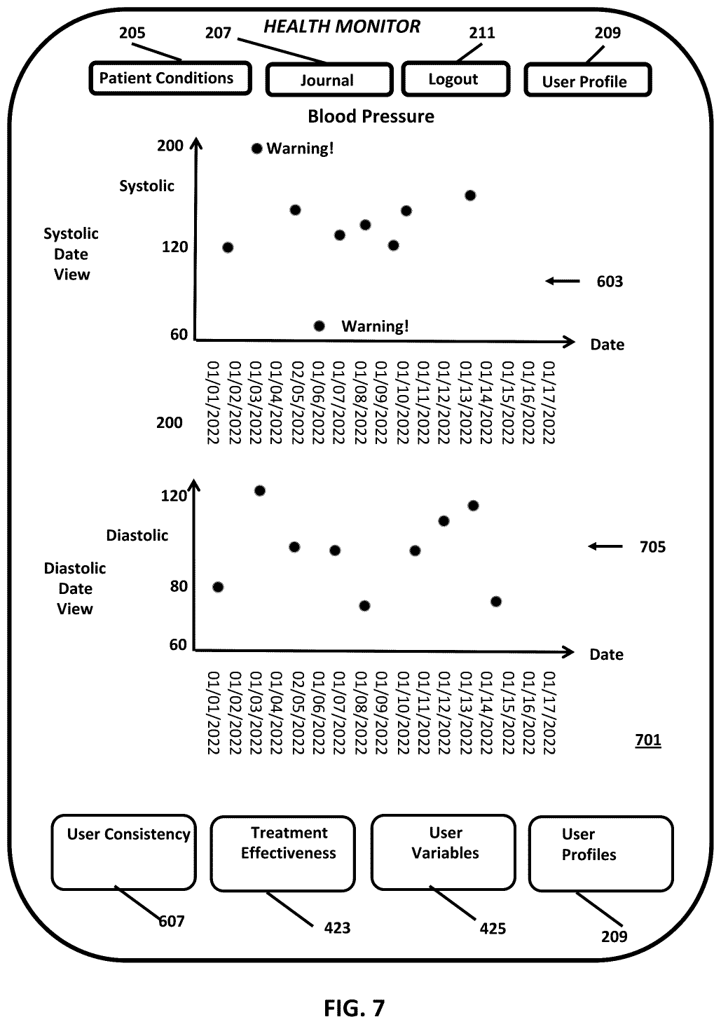

Clicking on Systolic/Diastolic button 605 results in page 701 shown in FIG. 7 being presented. Page 701 presents a graph 703 that illustrates systolic blood pressure readings as a function of time and a graph 705 that illustrates diastolic blood pressure readings as a function of time. The out of range blood pressure readings are flagged with a warning on graph 603 are flagged in graphs 703 and 705 as appropriate. The warnings are for two systolic readings and as such are flagged in graph 703.

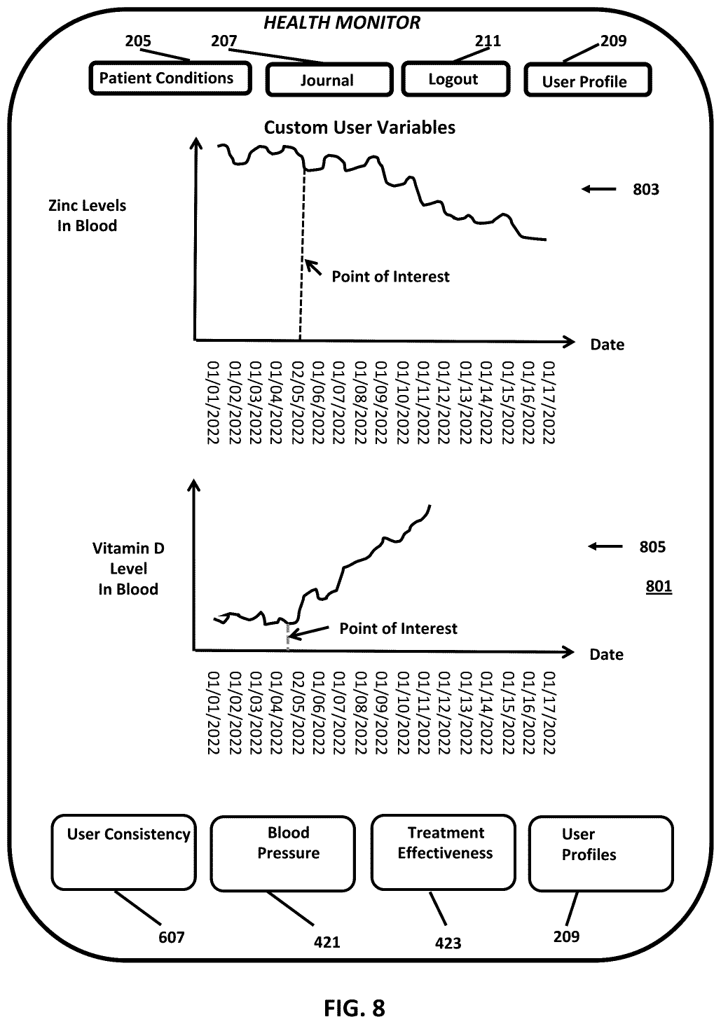

If the user clicks on User Variables button 425, page 801 shown in FIG. 8 is displayed. Page 801 shows graphs of customized user variables plotted against dates. In the embodiment the use has chosen to include levels of Zinc in the blood and also the level of Vitamin D in the blood. Graph 803 plots the Zinc level and Graph 805 plots the level of Vitamin D. In both graph 803 and graph 805, a point of interest is indicated that is obtained from an archive of journal conditions updated by date. Although only two graphs are shown of customized user variables, there may be other variables that the user is monitoring. Graphs for those other variables may be viewed by scrolling down the display of page 801.

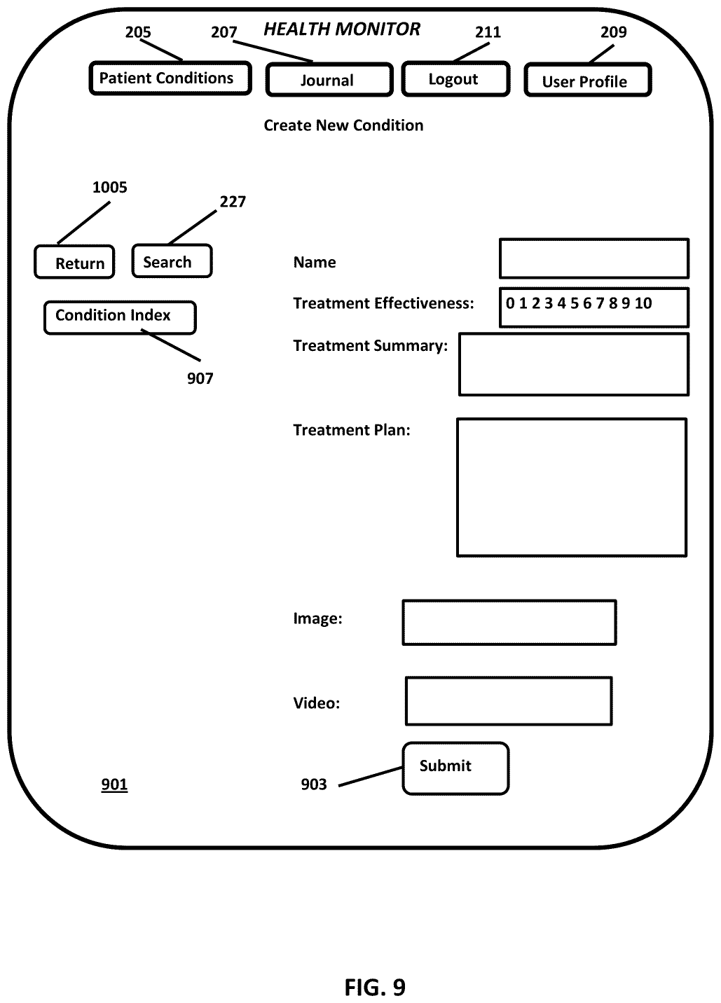

Clicking on Patient Conditions button 205 may bring up page 901 shown in FIG. 9 if there are no conditions previously entered. Page 901 permits the user to enter a new condition. A new condition is established by entering the name of the condition. If there is a treatment plan for that condition, a summary of the treatment is entered on page 901 and a description of the treatment is also entered. A relevant image of the condition and/or a video of the condition can be entered. After the information for the condition is entered on page 901, Submit button 903 may be clicked and the condition is saved.

Clicking on Conditions Index button 907 will cause page 201 shown in FIG.2 to be displayed

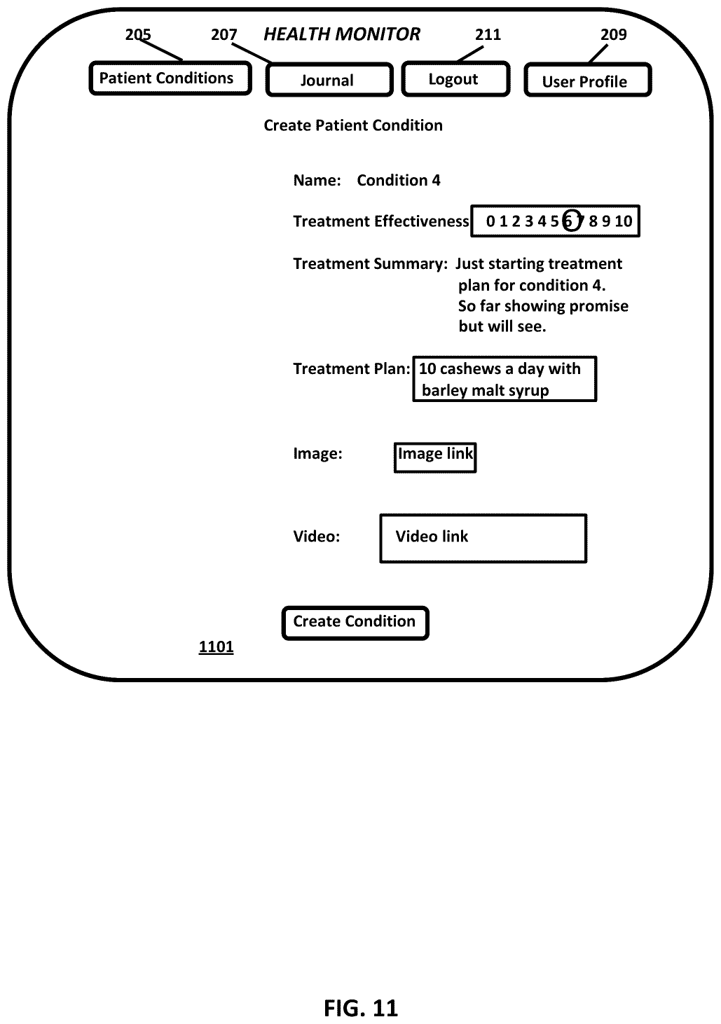

If it is desired to go directly to a specific condition, e.g. Condition 2, that condition may be entered into button 905. Clicking on button 905 after the condition has been entered will cause page 1001 shown in FIG. 10 to be displayed. Page 1001 shows the details that have been entered for Condition 2 selected from button 905. If it is desired to update one or more conditions, Update Condition button 1003 is clicked. If it is desired to create a new condition, Create New Condition button 229 is clicked and page 1101 shown in FIG. 11 is displayed.

On page 1101, the user enters the name of the condition, the treatment effectiveness, a summary of the treatment being used, a specific treatment plan, a link to an image of the condition, and a link to a video of the condition. Create Condition button 229 is clicked to create the condition.

Returning to page 1001 in FIG. 10, if the Update Condition button 1003 is clicked, page 1201 shown in FIG, 12 is displayed. On page 1201, each of Treatment Effectiveness 1203, Treatment Summary 1205, Treatment Plan 1207, Image link 1209, and Video link 1211 may be updated by clicking on Update Condition button 1213 or deleted in its entirety by clicking Delete Condition button 1215.

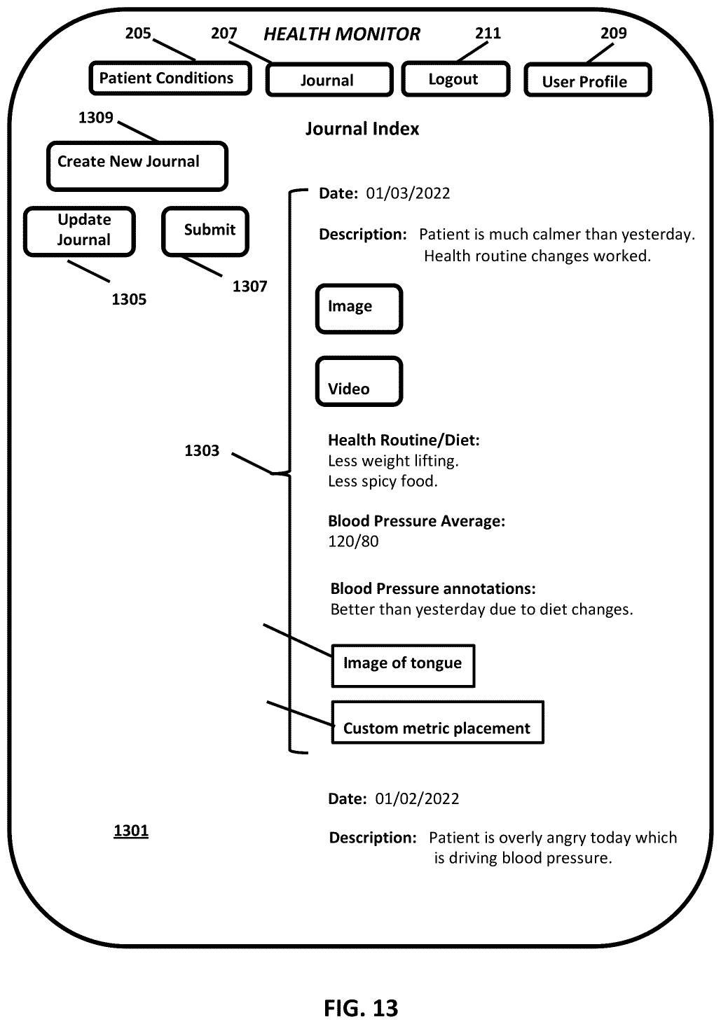

Clicking on Journal button 207 results in page 1301, shown in FIG.13 is displayed. Page 1301 displays a Journal Index starting with the most recent dated journal entry 1303. Scrolling down page 1301 will display other journal entries in reverse date order. Journal entry 1303 comprises the date of the journal entry, a Description, an Image, a Video, a description of the patient's Health Routine/Diet, the Blood Pressure Average for the day, any Blood Pressure Annotations, an Image of the patient's tongue, and Custom metric placement.

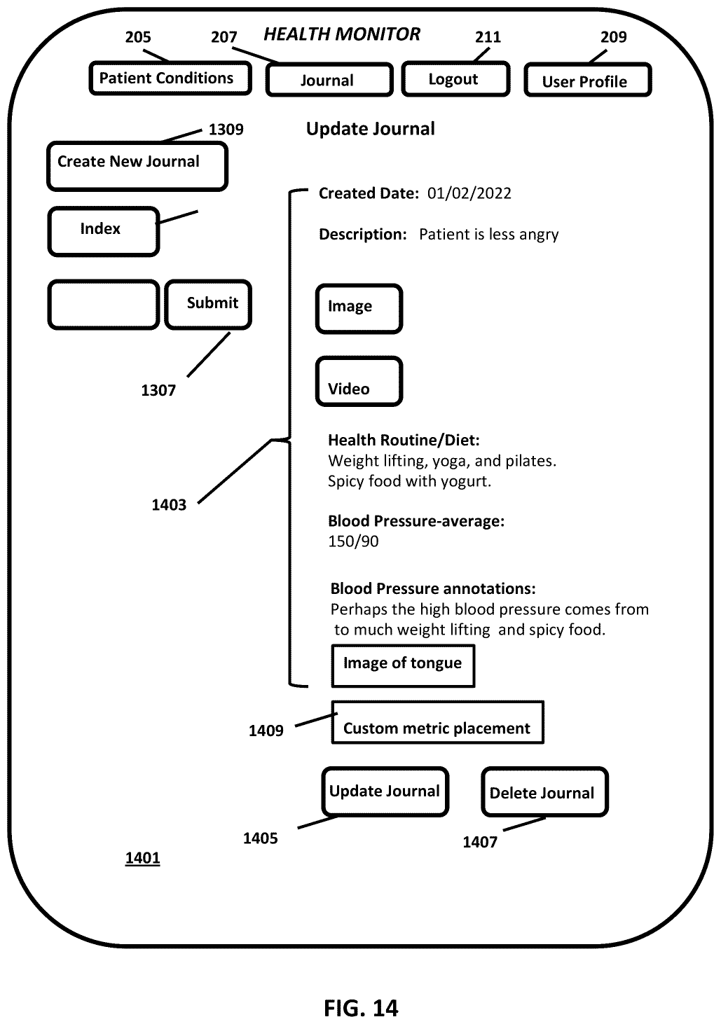

If it is desired to update the journal, Update Journal button 1305 is clicked. System 100 will display page 1401 shown in FIG. 14.

At page 1401, the user may enter the entry creation date, a description of the patient's condition, an image of the condition, a video of the condition, a health routine/diet followed by the patient, the patient's blood pressure, annotations to the blood pressure, and an image of the patient's tongue.

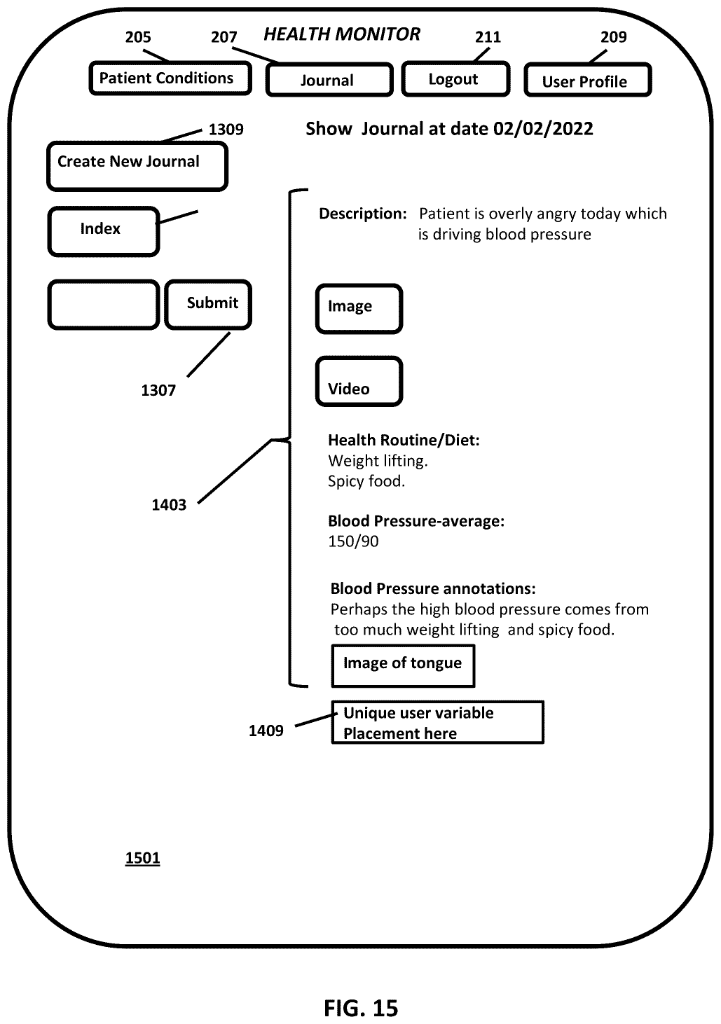

After the user has completed the entries, the user may click Update Journal button 1405, and the entry is complete and system 100 displays page 1501 shown in FIG. 15.

Alternatively, the user may click the Delete Journal button 1407 to delete the entry.

The user may add a custom metric by clicking on Custom Metric Placement button 1409.

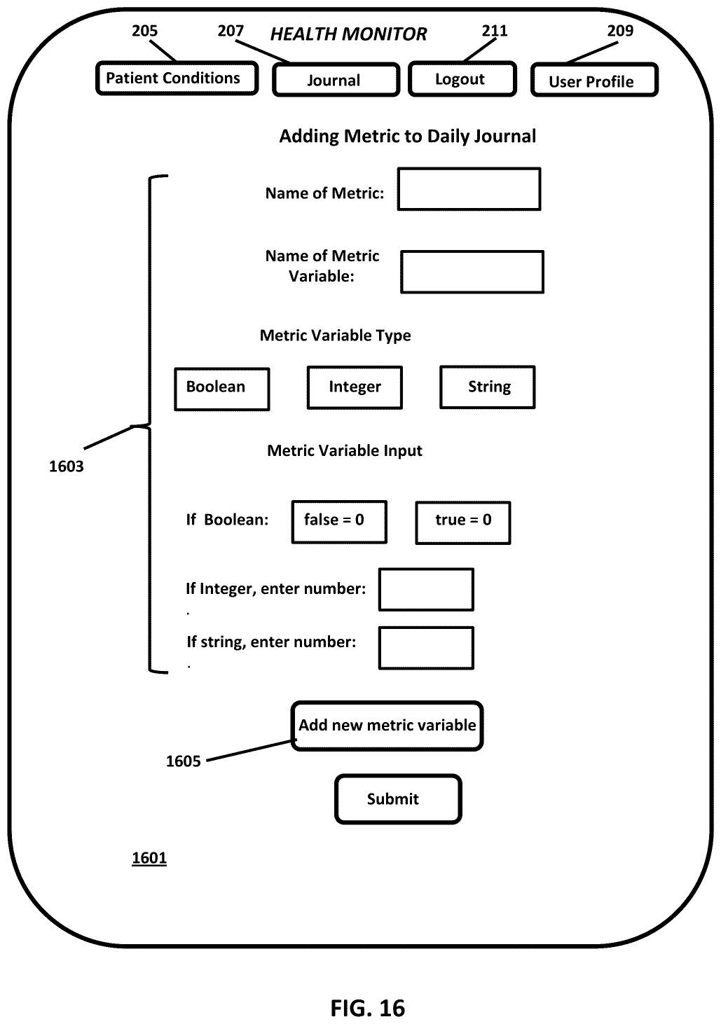

By clicking Custom Metric Placement button 1409, system 100 displays page 1601 shown in FIG. 16. At page 1601 a new metric may be entered. Clicking on Add New Metric button 1605 enters the new user selected metric to the daily journal.

The invention has been described in terms of specific embodiments. It will be appreciated by those skilled in the art that various changes and modifications may be made to the embodiments without departing from the scope of the invention. It is not intended that the invention be limited to the embodiments shown and described. It is intended that the invention include all foreseeable modifications to the embodiments shown and described. It is intended that the invention be limited in scope only by the claims appended hereto.

What is claimed is:

1. A health monitoring system that permits daily tracking of medical conditions and journaling of information pertaining to treatments of said conditions and providing a graphical user interface to see overall progress related to said daily tracking and journaling, comprising:
a processor;
a display coupled to said processor;
input apparatus for providing inputs to said processor;
a wireless interface for providing said processor with wireless access to a network;
program memory coupled to said processor;
a non-transient memory accessible by said processor, said non-transient memory comprising program instructions executable by said processor;
said program instructions comprising a conditions schema, a journal schema, and a user schema;
a data tracking database for use by said processor;
a conditions database for use by said processor;
said conditions schema including instructions to receive, via said input apparatus, entered health conditions, commentary on said patient's conditions, and commentary on treatment for said conditions, said conditions schema storing said health conditions, patient condition commentary, and treatment commentary in said condition database and generating a conditions table in a separate page, said conditions page comprising a treatment plan and a retrospect of past treatments;
said journaling schema comprising instructions for receiving daily journaling entries in said data tracking database and generating a journaling page in said data tracking database; and
said user schema comprising instructions to automatically generate graphical representations from said conditions schema and said journals schema and to show said graphical representations on said display upon request from a system user.

2. A health monitoring system in accordance with claim 1, wherein:
said user schema instructions executable by said processor utilize said data tracking database and said conditions database to said track user consistency with the treatment plans and to generate a graphical representation of said user consistency; and
said processor is operable to show said graphical representation of user consistency on said display.

3. A health monitoring system in accordance with claim 2, wherein:
said processor is operable to transmit said graphical representation of user consistency to a third party via said wireless interface.

4. A health monitoring system in accordance with claim 2, wherein:
said user schema instructions are executable by said processor automatically generate a graphical representation showing effectiveness of said treatment plans.

5. A health monitoring system in accordance with claim 4, wherein:
said processor is operable to selectively transmit said graphical representation showing effectiveness and said graphical representation of user consistency via said wireless interface.

6. A health monitoring system in accordance with claim 1, wherein:
said user schema instructions are executable to automatically generate and to visually display graphical representations of said conditions and treatment progression; and
said graphical representations show how said patient's condition is changing and provide visualization of amelioration of improvements and quantifiable aspects of said journal entries and said conditions via a graphical user interface.

7. A health monitoring system in accordance with claim 1, wherein:
said journaling schema instructions are executable to receive and store user created variables within said journaling page;
said processor is operable to display said user created variables; and
said user schema instructions are executable to create one of more graphical representations of said user created variables.

8. A health monitoring system in accordance with claim 7, wherein:
said user schema comprising instructions are executable to permit a user to check whether one of said one or more health conditions is improving or not; and
comprising instructions to generate graphical representations from said user selected variables.

9. A health monitoring system in accordance with claim 1, wherein:
said journaling schema instructions are executable to receive and store user created variables within said journaling page;
said processor is operable to display said user created variables; and
said user schema instructions are executable to access said stored user created variable to generate one of more graphical representations of said user created variables.

10. A health monitoring system in accordance with claim 1, wherein:
said program instructions are executable on said processor to track an index of said conditions in said conditions page separate from daily journaling page.

11. A health monitoring system in accordance with claim 1, wherein:
said program instructions are executable to display a list of conditions that a user can refer to with respect to a treatment plan and its effectiveness.

12. A health monitoring system in accordance with claim 1, wherein:
said executable program instructions generate one or more tables from a union of said conditions table and said journaling table to permit determining which of said conditions are improving.

13. A health monitoring system in accordance with claim 1, wherein:
said conditions schema provides a ranking scale responsive to said user to rank a treatment; and
said program instructions utilizes said ranking scale to assist said user to determine whether or not a new treatment plan is needed.

14. A health monitoring system in accordance with claim 1, wherein:
said journaling schema comprise program instructions executable to cause said system to be responsive to said user entering a description of the treatment plan to store and display said treatment plan;
said program instructions responsive to said user entering one or more links to images and videos of said condition to store and display said images and videos.

15. A health monitoring system in accordance with claim 1, wherein:
said system combines functionality from said journal schema, said conditions schemas, and said user schema to automatically produce graphical representations from said conditions schema.

16. A health monitoring system in accordance with claim 1, wherein:
said graphical representations include automatically generated graphs of user consistency, of patient blood pressure, and of user selected variables; and
said processor provides a graphic user interface to visualize said graphical representations.

17. A non-transient memory comprising program instructions executable by a processor to provide a health monitoring system that permits daily tracking of medical conditions and journaling of information pertaining to treatments of said conditions and providing a graphical user interface to see overall progress related to said daily tracking and journaling, comprising:
said program instructions comprising a conditions schema, a journal schema, and a user schema;
a data tracking database for use by said processor;
a conditions database for use by said processor;
said conditions schema including instructions to receive, via said input apparatus, entered health conditions, commentary on said patient's conditions, and commentary on treatment for said conditions, said conditions schema storing said health conditions, patient condition commentary, and treatment commentary is said condition database and generating a conditions table in a separate page, said conditions page comprising a treatment plan and a retrospect of past treatments;
said journaling schema comprising instructions for receiving daily journaling entries in said data tracking database and generating a journaling page in said data tracking database; and
said user schema comprising instructions to automatically generate graphical representations from said conditions schema and said journals schema and displaying said graphical representations on said display.

18. A non-transient memory comprising program instructions, in accordance with claim 17, wherein:
said user schema instructions executable by said processor utilize said data tracking database and said conditions database to said track user consistency with the treatment plans and to automatically generate a graphical representation of said user consistency.

19. A non-transient memory comprising program instructions, in accordance with claim 17, comprising:
user schema instructions executable by said processor to automatically generate a graphical representation showing effectiveness of said treatment plans

20. A non-transient memory comprising program instructions, in accordance with claim 17, comprising:
schema instructions executable to automatically generate graphical representations of said conditions and treatment progression; and
said graphical representations showing how said patient's condition is changing and providing visualization of amelioration of improvements and quantifiable aspects of said journal entries and said conditions via a graphical user interface.

21. A non-transient memory comprising program instructions, in accordance with claim 17, wherein:
said journaling schema instructions are executable to receive and store user created variables within said journaling page; and
said user schema instructions are executable to create one of more graphical representations of said user created variables.

22. A non-transient memory comprising program instructions, in accordance with claim 21, wherein:
said user schema comprises instructions are executable to permit a user to check whether one of said one or more health conditions is improving or not and instructions to generate graphical representations from said user selected variables.

23. A non-transient memory comprising program instructions, in accordance with claim 17, wherein:
said journaling schema instructions are executable to receive and store user created variables within said journaling page; and
said user schema instructions are executable to access said stored user created variable to generate one of more graphical representations of said user created variables.

24. A non-transient memory comprising program instructions, in accordance with claim 17, wherein:
said program instructions are executable on said processor to track an index of said conditions in said conditions page separate from daily journaling page.

25. A non-transient memory comprising program instructions, in accordance with claim 17, wherein:
said program instructions are executable to display a list of conditions that a user can refer to with respect to a treatment plan and its effectiveness.

26. A non-transient memory comprising program instructions, in accordance with claim 17, wherein:
said executable program instructions generate one or more tables from a union of said conditions table and said journaling table to permit determining which of said conditions are improving.

27. A non-transient memory comprising program instructions, in accordance with claim 17, wherein:
said conditions schema provides a ranking scale responsive to said user to rank a treatment; and
said program instructions utilize said ranking scale to assist said user to determine whether or not a new treatment plan is needed.

28. A non-transient memory comprising program instructions, in accordance with claim 17, wherein:
said journaling schema comprise program instructions executable to be responsive to said user entering a description of the treatment plan to store and display said treatment plan; and
said program instructions are responsive to said user entering one or more links to images and videos of said condition to store and display said images and videos.

29. A non-transient memory comprising program instructions, in accordance with claim 17, wherein:
said graphical representations include automatically generated graphs of user consistency, of patient blood pressure, and of user selected variables; and
said processor provides a graphic user interface to visualize said graphical representations.
